(19) United States
(12) Patent Application Publication
(10) Pub. No.: US 2025/0145573 A1
(43) Pub. Date: May 8, 2025

(54) COMPOSITION FOR PREVENTING, ALLEVIATING OR TREATING CANCER

(71) Applicant: CKP Therapeutics, Inc., Lowell, MA (US)

(72) Inventors: Ki Cheong PARK, Lowell, MA (US); Seokmo KIM, Lowell, MA (US)

(21) Appl. No.: 18/386,985

(22) Filed: Nov. 3, 2023

(51) Int. Cl. C07D 215/12; A61K 45/06; A61P 35/00

(52) U.S. Cl. CPC C07D 215/12; A61K 45/06; A61P 35/00

(57) ABSTRACT

A compound represented by Formula 1 according to the present disclosure, when used in combination with an anticancer drug, may significantly improve the anticancer effect of the anticancer drug, and may induce the same anticancer effect even when the anticancer drug is used in a significantly smaller amount than the conventionally used amount, thereby reducing the side effects caused by administration of the anticancer drug. Furthermore, the compound represented by Formula 1 makes it possible to effectively prevent, alleviate or treat either anticancer-resistant cancer or cancer which recurs or metastasizes after anticancer drug treatment.

### TECHNICAL FIELD

The present disclosure relates to a composition for preventing, alleviating or treating cancer.

### BACKGROUND ART

Cancer is a disease that contributes to a significant number of deaths worldwide, and cancer-related deaths worldwide in 2018 reached 9.6 million. In 1990s, cancer was the third leading cause of death, but in 2018, it ranked the second leading cause of death after heart diseases. Due to continued studies on cancer, the progression rate of cancer has decreased and the overall average survival rate has increased. Nevertheless, complete cure of cancer is still impossible due to resistance to anticancer drugs.

Anticancer drugs that are used in anticancer chemotherapy are drugs that inhibit the growth or proliferation of cancer cells. Anticancer drugs are roughly classified into cytotoxic anticancer drugs, targeted anticancer drugs, and immune anticancer drugs, and combination anticancer chemotherapy is also used in which anticancer drugs are selected depending on the type or degree of progression of cancer, the patient's condition, etc. and two or more drugs are used simultaneously to increase the effects thereof. Cytotoxic anticancer drugs, known as first-generation anticancer drugs, exhibit anticancer effects by directly attacking cells that differentiate indiscriminately and rapidly. However, these cytotoxic anticancer drugs have the disadvantage of causing side effects such as decreased leukocytes, hair loss, vomiting, diarrhea, etc. because they also attack normal cells having the property of rapidly differentiating, such as hair follicle cells. Targeted anticancer drugs, known as second-generation anticancer drugs, act on specific protein or specific gene changes that appear in cancer cells, thus blocking signaling involved in cancer growth and differentiation. Unlike cytotoxic anticancer agents, these targeted anticancer agents have fewer side effects because they specifically act only on cancer cells without acting on normal cells.

However, one of the obstacles to the use of anticancer chemotherapy is the occurrence of anticancer drug resistance. In order for anticancer chemotherapy for cancer patients to be successful, cancer cells must be killed at a blood concentration at which normal tissues can survive. The expression “resistance to an anticancer drug” refers to a case in which cancer cells are not killed even when the anticancer drug is administered in an amount that can reach a blood concentration at which cancer cells can be killed. Anticancer drug resistance may vary from patient to patient and may even be induced by various factors including genetic differences between tumors derived from the same tissue. Cancer cell types derived from a single patient can acquire different genetic characteristics, and show not only diversity of gene expression but also activation of tumor-inducing factors and inactivation of tumor suppressors, due to ‘mutation’. As a result, all types of cancers express anticancer drug resistance genes in different patterns, and cells in a single cancer mass acquire diversity of drug resistance. In addition, even though tumors are not originally resistant to a specific anticancer therapy, once they are exposed to an anticancer drug, cells resistant to the anticancer drug selectively grow based on this diversity, and eventually many cancer cells rapidly have anticancer drug resistance. In this case, if a plurality of drugs targeting different intracellular substances are used, these drugs can effectively treat cancer, and increase the cure rate of cancer. In many cases, however, cells are simultaneously resistant to drugs that are structurally or functionally completely different. This phenomenon is known as multi-drug resistance (MDR) and is caused by limiting the intracellular accumulation of drugs through limited absorption or increased release of anticancer drugs, or changes in membrane lipids such as ceramides. Multi-drug resistance inhibits apoptosis induced by most anticancer drugs, causes DNA damage repair and drug detoxification, and further imparts anticancer drug resistance to cells by changing the cell cycle.

Therefore, there is a need to develop a new anticancer drug that can not only kill cancer cells, but also induce the death of cancer cells with anticancer drug resistance.

### DISCLOSURE

### Technical Problem

An object of the present invention is to provide a pharmaceutical composition for co-administration for preventing or treating cancer.

Another object of the present invention is to provide a food composition for co-intake for preventing or treating cancer.

Still another object of the present invention is to provide a composition for preventing, alleviating or treating cancer.

Yet another object of the present invention is to provide a composition for enhancing sensitivity to anticancer drug treatment.

### Technical Solution

One embodiment of the present invention provides a pharmaceutical composition for administration in combination with an anticancer drug for preventing or treating cancer.

The pharmaceutical composition of the present invention contains a compound represented by the following Formula 1 or a pharmaceutically acceptable salt thereof as an active ingredient:

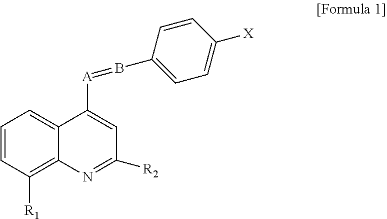

wherein
A and B are each independently —CH or N;
R₁ and R₂ are each independently selected from the group consisting of hydrogen and C₁-C₅ alkyl; and
X is hydrogen or halogen.

In the present invention, the term “halogen” refers to halogen group elements, including, for example, group 17 elements such as fluorine (F), chlorine (Cl), bromine (Br), and iodine (I).

In the compound represented by Formula 1 according to the present invention, A and B may each independently be —CH or N. Specifically, at least one of A and B may be —CH. More specifically, A and B may be different from each other.

In the compound represented by Formula 1 according to the present invention, R₁ and R₂ may each independently be selected from the group consisting of hydrogen and C₁-C₅ alkyl. Specifically, R₁ and R₂ may each independently be selected from the group consisting of C₁-C₃ alkyl. More specifically, R₁ and R₂ may each independently be C₁ alkyl, without being limited thereto.

In the compound represented by Formula 1 according to the present invention, X may be hydrogen or halogen. Specifically, the halogen may be fluorine (F) or chlorine (Cl). More specifically, the halogen may be chlorine (Cl), without being limited thereto.

In the present invention, the compound represented by Formula 1 may comprise a compound represented by Formula 2 or Formula 3 below:

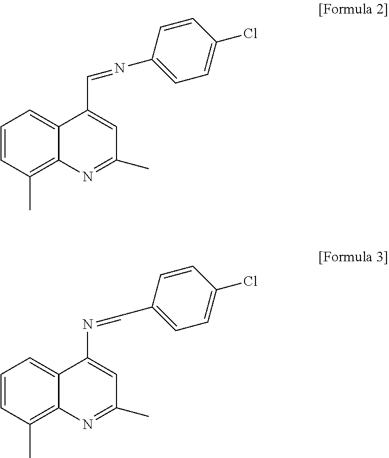 [SMILES: Cc1cc(C=Nc2ccc(Cl)cc2)c2cccc(C)c2n1 ; Cc1cc(N=Cc2ccc(Cl)cc2)c2cccc(C)c2n1]

In the present invention, the compound represented by Formula 2 or 3 may be CKP1 or CKP2, which is a candidate compound discovered through in silico screening in one example of the present invention.

In one embodiment of the present invention, the compound represented by Formula 2 or 3 is able to target SERCA (ATP2A1) protein. In particular, the compound represented by Formula 2 or 3 is able to specifically inhibit SERCA1 protein.

In the present invention, the term “pharmaceutically acceptable salt” refers to such salts which are usually considered by those skilled in the art to be suitable for medical applications, e.g., because they are not harmful to subjects which may be treated with the salts, or which give rise to side effects which are tolerable within the respective treatment. Usually, the pharmaceutically acceptable salts are such salts which are considered as acceptable by the regulatory authorities, such as the US Food and Drug Administration (FDA), the European Medicines Agency (EMA), or the Japanese Ministry of Health, Labor and Welfare Pharmaceuticals and Medical Devices Agency (PMDA). However, the present invention in principle also encompasses salts of the compounds according to the present invention which are as such not pharmaceutically acceptable, e.g., as intermediates in the production of the compounds according to the present invention or physiologically functional derivatives thereof, or as intermediates in the production of pharmaceutically acceptable salts of the compounds according to the present invention or physiologically functional derivatives thereof. Said salts include water-insoluble salts and, particularly, water-soluble salts.

In each case, those skilled in the art can readily determine whether a certain compound according to the present invention or a physiologically functional derivative thereof can form a salt, i.e., whether said compound according to the present invention or physiologically functional derivative thereof has a group which may carry a charge, such as, for example, an amino group, a carboxylic acid group, etc.

Exemplary salts of the compounds of the present invention are acid addition salts or salts with bases, particularly pharmaceutically acceptable inorganic and organic acid addition salts and salts with bases customarily used in pharmacy, which are either water insoluble or, particularly, water-soluble acid addition salts. Salts with bases may, depending on the substituents of the compounds of the present invention, also be suitable. Acid addition salts may, for example, be formed by mixing a solution of a compound of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid, or phosphoric acid. Likewise, pharmaceutically acceptable base addition salts may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, arginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, digluconate, dihydrochloride, dodecylsulfate, edetate, edisylate, ethanesulfonate, formate, fumarate, galactate, galacturonate, gluconate, glutamate, glycerophosphate, hemisulfate, heptanoate, hexanoate, hexylresorcinate, hydrobromide, hydrochloride, hydroiodide, 2-hydroxy-ethanesulfonate, hydroxynaphthoate, iodide, isobutyrate, isothionate, lactate, laurate, lauryl sulfate, malate, maleate, malonate, mandelate, methanesulfonate (mesylate), methylsulfate, 2-naphthalenesulfonate, nicotinate, nitrate, oleate, oxalate, palmitate, pantothenate, pectinate, persulfate, 3-phenylpropionate, phosphate/diphosphate, phthalate, picrate, pivalate, polygalacturonate, propionate, salicylate, stearate, sulfate, suberate, succinate, tannate, tartrate, tosylate, undecanoate, valerate, and the like.

Salts, which are not pharmaceutically acceptable and which can be obtained, for example, as process products during the production of the compounds according to the present invention on an industrial scale, are also encompassed by the present invention and, if desired, may be converted into pharmaceutically acceptable salts by processes known to those skilled in the art.

The cancer in the present invention may be resistant, recurrent or metastatic cancer. For the purposes of the present invention, the cancer may be cancer resistant to anticancer drug treatment, or may be cancer that metastasized or recurred after treatment. Here, the anticancer drug is not particularly limited in the kind thereof and may be any type of anticancer drug, but may specifically be a tyrosine kinase inhibitor (TKI), without being limited thereto.

The cancer in the present invention may be any one or more selected from the group consisting of thyroid cancer, colorectal cancer, breast cancer, uterine cancer, fallopian tube cancer, ovarian cancer, gastric cancer, brain cancer, rectal cancer, small intestine cancer, esophagus cancer, lymph node cancer, gallbladder cancer, lung cancer, skin cancer, kidney cancer, bladder cancer, blood cancer, pancreatic cancer, prostate cancer, endocrine gland cancer, oral cancer, and liver cancer. For example, the cancer may be thyroid cancer, without being limited thereto.

The compound represented by Formula 1 according to the present invention may be administered in combination with an anticancer drug to enhance the activity of the anticancer drug. In one example of the present invention, it was confirmed that, when the compound represented by Formula 1 was administered in combination with a tyrosine kinase inhibitor, particularly sorafenib or lenvatinib, which is a kind of anticancer drug, it could effectively enhance the activity of the anticancer drug in drug-resistant, recurrent or metastatic cancer, thereby very effectively reducing the size and weight of tumors.

The anticancer drug of the present invention may comprise any drug whose activity may be enhanced by the compound represented by Formula 1 according to the present invention to prevent or treat cancer. Here, the drug is not particularly limited in the kind thereof and may be any kind of drug that is capable of preventing or treating cancer. For example, the anticancer drug may be at least one selected from the group consisting of lenvatinib, sorafenib, imatinib, erlotinib, neratinib, lapatinib, gefitinib, vandetanib, nilotinib, semasanib, bosutinib, axitinib, cediranib, regorafenib, lestaurtinib, asciminib, ibrutinib, sunitinib, afatinib, dasatinib, nitrogen mustard, oxaliplatin, rituximab, panitumumab, trastuzumab, bortezomib, carboplatin, bevacizumab, cisplatin, cetuximab, aflibercept, Viscum album, asparaginase, hydroxycarbamide, estramustine, gemtuzumab ozogamicin, ibritumomab tiuxetan, heptaplatin, methylaminolevulinic acid, amsacrine, alemtuzumab, procarbazine, alprostadil, holmium nitrate chitosan, gemcitabine, doxifluridine, pemetrexed, tegafur, capecitabine, gimeracin, oteracil, azacitidine, methotrexate, cytarabine, fluorouracil, fludarabine, enocitabine, flutamide, decitabine, mercaptopurine, thioguanine, cladribine, leucovorin, carmophor, raltitrexed, interferon alpha-2a, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, pepromycin, temsirolimus, temozolomide, busulfan, ifosfamide, cyclophosphamide, melphalan, altretamine, dacarbazine, thiotepa, nimustine, chlorambucil, mitolactol, leucovorin, tretonin, exemestane, amino glutesimide, anagrelide, navelbine, fadrazole, tamoxifen, toremifene, testolactone, anastrozole, letrozole, vorozol, bicalutamide, lomustine, and carmustine. Specifically, the tyrosine kinase inhibitor may be at least one selected from the group consisting of lenvatinib, sorafenib, imatinib, erlotinib, neratinib, lapatinib, gefitinib, vandetanib, nilotinib, semasanib, bosutinib, axitinib, cediranib, regorafenib, lestaurtinib, asciminib, ibrutinib, sunitinib, afatinib, and dasatinib, without being limited thereto.

In the present invention, the term “prevention” or “preventing” refers to any action that suppresses or slows the onset of a disease or condition. For the purposes of the present invention, the term means that the composition is used in combination with an anticancer drug to slow or suppress the onset of cancer.

In the present invention, the term “treatment” or “treating” refers to any action that slows, halts or reverses the progression of a disease or condition. For the purposes of the present invention, the term means that the composition is used in combination with an anticancer drug to halt, alleviate, mitigate, eliminate or reverse the progression of cancer.

The pharmaceutical composition of the present invention may be in the form of capsules, tablets, granules, injections, ointments, powders, or beverages, and the pharmaceutical composition may be for administration to humans.

For use, the pharmaceutical composition of the present invention may be formulated in the form of oral preparations such as powders, granules, capsules, tablets, and aqueous suspensions, preparations for external use, suppositories, and sterile injectable solutions, according to the respective conventional methods, without being limited thereto. The pharmaceutical composition of the present invention may contain pharmaceutically acceptable carriers. As the pharmaceutically acceptable carriers, a binder, a lubricant, a disintegrant, an excipient, a solubilizer, a dispersant, a stabilizer, a suspending agent, a colorant, a flavoring agent, and the like may be used for oral administration; a buffer, a preservative, a pain-relieving agent, a solubilizer, an isotonic agent, a stabilizer, and the like may be used for injection; and a base, an excipient, a lubricant, a preservative, and the like may be used for topical administration.

The pharmaceutical composition of the present invention may be prepared in various dosage forms by being mixed with the pharmaceutically acceptable carriers as described above. For example, for oral administration, the pharmaceutical composition may be prepared in the form of tablets, troches, capsules, elixirs, suspensions, syrups, wafers, or the like. For injection, the pharmaceutical composition may be prepared in the form of unit dosage ampoules or in multiple-dosage forms. In addition, the pharmaceutical composition may be formulated into solutions, suspensions, tablets, capsules, sustained-release preparations, or the like.

Examples of carriers, excipients and diluents suitable for formulation include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil. In addition, the pharmaceutical composition of the present invention may further contain a filler, an anticoagulant, a lubricant, a wetting agent, a fragrance, an emulsifier, a preservative, or the like.

The routes of administration of the pharmaceutical composition according to the present invention include, but are not limited to, oral, intravenous, intramuscular, intra-arterial, intramedullary, intradural, intracardiac, transdermal, subcutaneous, intraperitoneal, intranasal, gastrointestinal, topical, sublingual and intrarectal routes. Oral or parenteral administration is preferred. In the present invention, “parenteral” includes subcutaneous, transdermal, intravenous, intramuscular, intra-articular, intra-synovial, intrasternal, intradural, intra-lesional and intra-cranial injection or infusion techniques. The pharmaceutical composition of the present invention may also be formulated as suppositories for intrarectal administration.

The dose of the pharmaceutical composition of the present invention may vary depending on various factors, including the activity of a specific compound used, the patient's age, body weight, general health status, sex, and diet, the time of administration, the route of administration, excretion rate, drug combination, and the severity of a particular disease to be prevented or treated. Although the dose of the pharmaceutical composition may vary depending on the patient's condition and body weight, the severity of the disease, the form of drug, and the route and duration of administration, it may be appropriately selected by those skilled in the art. The pharmaceutical composition may be administered at a dose of 0.0001 to 50 mg/kg/day or 0.001 to 50 mg/kg/day. The pharmaceutical composition may be administered once a day or several times a day. The dose does not limit the scope of the present invention in any way. The pharmaceutical composition according to the present invention may be formulated as pills, sugar-coated tablets, capsules, liquids, gels, syrups, slurries, or suspensions.

Another embodiment of the present invention provides a food composition for intake in combination with an anticancer drug for prevention or alleviation of cancer.

The food composition of the present invention contains a compound represented by the following Formula 1 or a pharmaceutically acceptable salt thereof as an active ingredient:

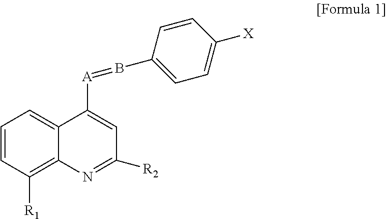

wherein
A and B are each independently —CH or N;
R₁ and R₂ are each independently selected from the group consisting of hydrogen and C₁-C₅ alkyl; and
X is hydrogen or halogen.

In the food composition of the present invention, contents regarding the compound represented by Formula 1, pharmaceutically acceptable salt, cancer, anticancer drug, etc. are the same as those described above, and thus description thereof will be omitted.

In the present invention, the term “alleviation” or “alleviating” refers to any action that alleviates or beneficially changes a disease or condition. For the purposes of the present invention, the term means that the composition is used in combination with an anticancer drug to alleviate the symptoms of cancer.

The food composition of the present invention may be prepared in the form of various foods, for example, beverages, gums, teas, vitamin complexes, powders, granules, tablets, capsules, confectionery, cakes, bread, etc.

When the compound represented by Formula 1 according to the present invention or a pharmaceutically acceptable salt thereof is contained as an active ingredient in a food composition, it may be added in an amount of 0.1 to 50 wt % based on the total weight of the food composition, without being limited thereto.

When the food composition of the present invention is prepared as a beverage, there is no particular limitation, except that the beverage contains the food composition at the indicated percentage. In this case, the beverage may additionally contain various flavorings or natural carbohydrates, like conventional beverages. Specific examples of the natural carbohydrates include monosaccharides such as glucose, disaccharides such as fructose, polysaccharides such as sucrose, conventional sugars such as dextrin, cyclodextrin or the like, and sugar alcohols such as xylitol, sorbitol, erythritol or the like. Examples of the flavorings include natural flavorings (thaumatin, stevia extracts, such as rebaudioside A, glycyrrhizin, etc.) and synthetic flavorings (saccharin, aspartame, etc.).

The food composition of the present invention may further contain various nutrients, vitamins, minerals (electrolytes), flavorings such as synthetic flavorings and natural flavorings, colorants, pectic acid and its salt, alginic acid and its salt, organic acids, protective colloidal thickeners, pH adjusting agents, stabilizers, preservatives, glycerin, alcohol, carbonizing agents that are used in carbonated beverages, etc.

The components contained in the food composition of the invention may be used independently or in combination. Although the content of the additives in the food composition is not critical to the present invention, it may be selected in the range of 0.1 to about 50 parts by weight based on 100 parts by weight of the food composition of the present invention, without being limited thereto.

Still another embodiment of the present invention provides a composition for preventing, alleviating or treating cancer.

The composition of the present invention contains a compound represented by the following Formula 1 or a pharmaceutically acceptable salt thereof as an active ingredient:

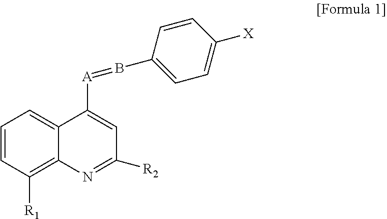

wherein
A and B are each independently —CH or N;
R₁ and R₂ are each independently selected from the group consisting of hydrogen and C₁-C₅ alkyl; and
X is hydrogen or halogen.

In the present invention may further contain an anticancer drug whose activity may be enhanced by the compound represented by Formula 1 according to the present invention to prevent or treat cancer. The anticancer drug may be, for example, at least one selected from the group consisting of lenvatinib, sorafenib, imatinib, erlotinib, neratinib, lapatinib, gefitinib, vandetanib, nilotinib, semasanib, bosutinib, axitinib, cediranib, regorafenib, lestaurtinib, asciminib, ibrutinib, sunitinib, afatinib, dasatinib, nitrogen mustard, oxaliplatin, rituximab, panitumumab, trastuzumab, bortezomib, carboplatin, bevacizumab, cisplatin, cetuximab, aflibercept, viscum album, asparaginase, hydroxycarbamide, estramustine, gemtuzumab ozogamicin, ibritumomab tiuxetan, heptaplatin, methylaminolevulinic acid, amsacrine, alemtuzumab, procarbazine, alprostadil, holmium nitrate chitosan, gemcitabine, doxifluridine, pemetrexed, tegafur, capecitabine, gimeracin, oteracil, azacitidine, methotrexate, cytarabine, fluorouracil, fludarabine, enocitabine, flutamide, decitabine, mercaptopurine, thioguanine, cladribine, leucovorin, carmophor, raltitrexed, interferon alpha-2a, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, pepromycin, temsirolimus, temozolomide, busulfan, ifosfamide, cyclophosphamide, melphalan, altretamine, dacarbazine, thiotepa, nimustine, chlorambucil, mitolactol, leucovorin, tretonin, exemestane, amino glutesimide, anagrelide, navelbine, fadrazole, tamoxifen, toremifene, testolactone, anastrozole, letrozole, vorozol, bicalutamide, lomustine, and carmustine. Specifically, the tyrosine kinase inhibitor may be, for example, at least one selected from the group consisting of lenvatinib, sorafenib, imatinib, erlotinib, neratinib, lapatinib, gefitinib, vandetanib, nilotinib, semasanib, bosutinib, axitinib, cediranib, regorafenib, lestaurtinib, asciminib, ibrutinib, sunitinib, afatinib, and dasatinib, without being limited thereto.

In the composition of the present invention, the compound and the anticancer drug may be used at a ratio of 1:0.001 to 1:1000, preferably 1:0.01 to 1:100, more preferably 1:0.1 to 1:10, without being limited thereto. Here, the ratio may be a molar concentration ratio or a weight ratio, without being limited thereto.

In the composition for preventing, alleviating or treating cancer according to the present invention, details regarding the compound represented by Formula 1 or Formula 2, pharmaceutically acceptable salt, and cancer overlaps with those described above, and thus detailed description thereof will be omitted below.

In the present invention, the term “prevention” or “preventing” refers to any action that suppresses or slows the onset of a disease or condition. For the purposes of the present invention, the term means that the composition is used in combination with an anticancer drug to slow or suppress the onset of cancer.

In the present invention, the term “alleviation” or “alleviating” refers to any action that alleviates or beneficially changes a disease or condition. For the purposes of the present invention, the term means that the composition is used in combination with an anticancer drug to alleviate the symptoms of cancer.

In the present invention, the term “treatment” or “treating” refers to any action that slows, halts or reverses the progression of a disease or condition. For the purposes of the present invention, the term means that the composition is used in combination with an anticancer drug to halt, alleviate, mitigate, eliminate or reverse the progression of cancer.

The composition of the present invention may be used as a pharmaceutical composition or a food composition, and the form thereof is not particularly limited.

The pharmaceutical composition of the present invention may be in the form of capsules, tablets, granules, injections, ointments, powders, or beverages, and the pharmaceutical composition may be for administration to humans.

For use, the pharmaceutical composition of the present invention may be formulated in the form of oral preparations such as powders, granules, capsules, tablets, and aqueous suspensions, preparations for external use, suppositories, and sterile injectable solutions, according to the respective conventional methods, without being limited thereto. The pharmaceutical composition of the present invention may contain pharmaceutically acceptable carriers. As the pharmaceutically acceptable carriers, a binder, a lubricant, a disintegrant, an excipient, a solubilizer, a dispersant, a stabilizer, a suspending agent, a colorant, a flavoring agent, and the like may be used for oral administration; a buffer, a preservative, a pain-relieving agent, a solubilizer, an isotonic agent, a stabilizer, and the like may be used for injection; and a base, an excipient, a lubricant, a preservative, and the like may be used for topical administration.

The pharmaceutical composition of the present invention may be prepared in various dosage forms by being mixed with the pharmaceutically acceptable carriers as described above. For example, for oral administration, the pharmaceutical composition may be prepared in the form of tablets, troches, capsules, elixirs, suspensions, syrups, wafers, or the like. For injection, the pharmaceutical composition may be prepared in the form of unit dosage ampoules or in multiple-dosage forms. In addition, the pharmaceutical composition may be formulated into solutions, suspensions, tablets, capsules, sustained-release preparations, or the like.

Examples of carriers, excipients and diluents suitable for formulation include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil. In addition, the pharmaceutical composition of the present invention may further contain a filler, an anticoagulant, a lubricant, a wetting agent, a fragrance, an emulsifier, a preservative, or the like.

The routes of administration of the pharmaceutical composition according to the present invention include, but are not limited to, oral, intravenous, intramuscular, intra-arterial, intramedullary, intradural, intracardiac, transdermal, subcutaneous, intraperitoneal, intranasal, gastrointestinal, topical, sublingual and intrarectal routes. Oral or parenteral administration is preferred. In the present invention, “parenteral” includes subcutaneous, transdermal, intravenous, intramuscular, intra-articular, intra-synovial, intrasternal, intradural, intra-lesional and intra-cranial injection or infusion techniques. The pharmaceutical composition may also be formulated as suppositories for intrarectal administration.

The dose of the pharmaceutical composition of the present invention may vary depending on various factors, including the activity of a specific compound used, the patient's age, body weight, general health status, sex, and diet, the time of administration, the route of administration, excretion rate, drug combination, and the severity of a particular disease to be prevented or treated. Although the dose of the pharmaceutical composition may vary depending on the patient's condition and body weight, the severity of the disease, the form of drug, and the route and duration of administration, it may be appropriately selected by those skilled in the art. The pharmaceutical composition may be administered at a dose of 0.0001 to 50 mg/kg/day or 0.001 to 50 mg/kg/day. The pharmaceutical composition may be administered once a day or several times a day. The dose does not limit the scope of the present invention in any way. The pharmaceutical composition according to the present invention may be formulated as pills, sugar-coated tablets, capsules, liquids, gels, syrups, slurries, or suspensions.

The food composition of the present invention may be prepared in the form of various foods, for example, beverages, gums, teas, vitamin complexes, powders, granules, tablets, capsules, confectionery, cakes, bread, etc.

When the compound represented by Formula 1 according to the present invention or a pharmaceutically acceptable salt thereof is contained as an active ingredient in a food composition, it may be added in an amount of 0.1 to 50 wt % based on the total weight of the food composition, without being limited thereto.

When the food composition of the present invention is prepared as a beverage, there is no particular limitation, except that the beverage contains the food composition at the indicated percentage. In this case, the beverage may additionally contain various flavorings or natural carbohydrates, like conventional beverages. Specific examples of the natural carbohydrates include monosaccharides such as glucose, disaccharides such as fructose, polysaccharides such as sucrose, conventional sugars such as dextrin, cyclodextrin or the like, and sugar alcohols such as xylitol, sorbitol, erythritol or the like. Examples of the flavorings include natural flavorings (thaumatin, stevia extracts, such as rebaudioside A, glycyrrhizin, etc.) and synthetic flavorings (saccharin, aspartame, etc.).

The food composition of the present invention may further contain various nutrients, vitamins, minerals (electrolytes), flavorings such as synthetic flavorings and natural flavorings, colorants, pectic acid and its salt, alginic acid and its salt, organic acids, protective colloidal thickeners, pH adjusting agents, stabilizers, preservatives, glycerin, alcohol, carbonizing agents that are used in carbonated beverages, etc.

The components contained in the food composition of the invention may be used independently or in combination. Although the content of the additives in the food composition is not critical to the present invention, but may be selected in the range of 0.1 to about 50 parts by weight based on 100 parts by weight of the food composition of the present invention, without being limited thereto.

Yet another embodiment of the present invention provides a pharmaceutical composition for enhancing sensitivity to anticancer drug treatment, which serves to treat anticancer drug resistance or enhance anticancer drug sensitivity.

The pharmaceutical composition of the present invention contains a compound represented by the following Formula 1 or a pharmaceutically acceptable salt thereof as an active ingredient:

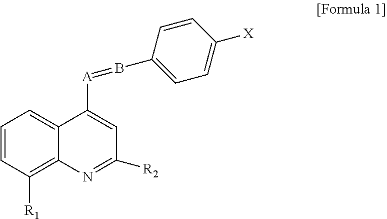

wherein
A and B are each independently —CH or N;
R₁ and R₂ are each independently selected from the group consisting of hydrogen and C₁-C₅ alkyl; and
X is hydrogen or halogen.

In the pharmaceutical composition of the present invention, contents regarding the compound represented by Formula 1, pharmaceutically acceptable salt, anticancer drug, etc., are the same as those described above, and thus description thereof will be omitted.

In the present invention, the term “anticancer drug resistance” refers to a condition in which the effect of an anticancer drug decreases when the anticancer drug is used repeatedly in a fixed amount, or a condition in which the amount or frequency of use of an anticancer drug needs to be increased in order to obtain the same effect as previously experienced by a patient with anticancer drug resistance, or a condition in which the same effect as before is not obtained even when the same dose of an anticancer drug as before is administered.

In the present invention, the phrase “treating anticancer drug resistance” refers to an action of recovering from a condition in which the effect of an anticancer drug decreases when the anticancer drug is used repeatedly in a fixed amount, or a condition in which the amount or frequency of use of an anticancer drug needs to be increased in order to obtain the same effect as previously experienced by a patient with anticancer drug resistance, or a condition in which the same effect as before is not obtained even when the same dose of an anticancer drug as before is administered. More specifically, the phrase “treating anticancer drug resistance” refers to an action of making an anticancer drug exhibit the same anticancer effect even when the anticancer drug is applied less frequently or at lower doses, or an action of returning to a state prior to the occurrence of anticancer drug resistance so that the same effect can be obtained even when the same or lower dose of an anticancer drug as before is administered.

In the present invention, the anticancer drug may be at least one selected from the group consisting of lenvatinib, sorafenib, imatinib, erlotinib, neratinib, lapatinib, gefitinib, vandetanib, nilotinib, semasanib, bosutinib, axitinib, cediranib, regorafenib, lestaurtinib, asciminib, ibrutinib, sunitinib, afatinib, dasatinib, nitrogen mustard, oxaliplatin, rituximab, panitumumab, trastuzumab, bortezomib, carboplatin, bevacizumab, cisplatin, cetuximab, aflibercept, Viscum album, asparaginase, hydroxycarbamide, estramustine, gemtuzumab ozogamicin, ibritumomab tiuxetan, heptaplatin, methylaminolevulinic acid, amsacrine, alemtuzumab, procarbazine, alprostadil, holmium nitrate chitosan, gemcitabine, doxifluridine, pemetrexed, tegafur, capecitabine, gimeracin, oteracil, azacitidine, methotrexate, cytarabine, fluorouracil, fludarabine, enocitabine, flutamide, decitabine, mercaptopurine, thioguanine, cladribine, leucovorin, carmophor, raltitrexed, interferon alpha-2a, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, pepromycin, temsirolimus, temozolomide, busulfan, ifosfamide, cyclophosphamide, melphalan, altretamine, dacarbazine, thiotepa, nimustine, chlorambucil, mitolactol, leucovorin, tretonin, exemestane, amino glutesimide, anagrelide, navelbine, fadrazole, tamoxifen, toremifene, testolactone, anastrozole, letrozole, vorozol, bicalutamide, lomustine, and carmustine. Specifically, the anticancer drug may be at least one selected from the group consisting of lenvatinib, sorafenib, imatinib, erlotinib, neratinib, lapatinib, gefitinib, vandetanib, nilotinib, semasanib, bosutinib, axitinib, cediranib, regorafenib, lestaurtinib, asciminib, ibrutinib, sunitinib, afatinib, and dasatinib. More specifically, the anticancer drug may be lenvatinib or sorafenib, but is not limited thereto and may be any drug that inhibits tyrosine kinase.

The composition of the present invention is capable of effectively treating anticancer drug-resistant cancer. The composition of the present invention is capable of increasing anticancer drug sensitivity while lowering the anticancer drug resistance of anticancer-resistant cancer, and thus may be very effectively used for the prevention, alleviation or treatment of cancer.

Still yet another embodiment of the present invention provides a method for preventing, alleviating or treating cancer.

The method of the present invention comprises a step of administering an effective amount of a compound represented by the following Formula 1 or a pharmaceutically acceptable salt thereof to a subject in need thereof:

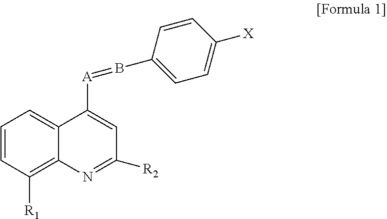

wherein
A and B are each independently —CH or N;
R₁ and R₂ are each independently selected from the group consisting of hydrogen and C₁-C₅ alkyl; and
X is hydrogen or halogen.

A further embodiment of the present invention provides a method of overcoming or treating anticancer drug resistance or enhancing anticancer drug sensitivity.

The method of the present invention comprises a step of administering an effective amount of a compound represented by the following Formula 1 or a pharmaceutically acceptable salt thereof to a subject in need thereof:

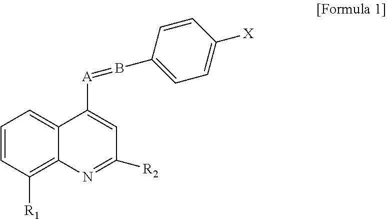

wherein
A and B are each independently —CH or N;
R₁ and R₂ are each independently selected from the group consisting of hydrogen and C₁-C₅ alkyl; and
X is hydrogen or halogen.

In the method for preventing, alleviating or treating cancer according to the present invention and the method for overcoming or treating anticancer drug resistance or enhancing anticancer drug sensitivity according to the present invention, contents regarding the compound represented by Formula 1, pharmaceutically acceptable salt, anticancer drug, etc. are the same as those described above, and thus description thereof will be omitted.

In the present invention, the term “administering” means providing a given composition of the present invention to a subject by any suitable method.

In the present invention, the phrase “subject in need thereof” may include both mammals and non-mammals. Here, examples of the mammals include, but are not limited to, humans, non-human primates such as chimpanzees, other ape or monkey species; livestock animals such as cattle, horses, sheep, goats, and pigs; domesticated animals such as rabbits, dogs or cats; laboratory animals, for example, rodents such as rats, mice, or guinea pigs. In addition, examples of the non-mammals in the present invention include, but are not limited to, birds or fish.

In the present invention, the formulation of the composition that is administered as described above is not particularly limited, and may be administered as solid form preparations, liquid form preparations, or aerosol preparations for inhalation. Specifically, the composition may be administered as solid form preparations which are intended to be converted, shortly before use, to liquid form preparations for oral or parenteral administration. For example, the composition may be formulated and administered as oral dosage forms, including powders, granules, capsules, tablets, and aqueous suspensions, preparations for external use, suppositories, and sterile injectable solutions, without being limited thereto.

In addition, in the present invention, pharmaceutically acceptable carriers may be additionally administered together with the composition of the present invention. Here, as the pharmaceutically acceptable carriers, a binder, a lubricant, a disintegrant, an excipient, a solubilizer, a dispersant, a stabilizer, a suspending agent, a colorant, a flavoring agent, and the like may be used for oral administration; a buffer, a preservative, a pain-relieving agent, a solubilizer, an isotonic agent, a stabilizer, and the like may be used for injection; and a base, an excipient, a lubricant, a preserving agent, and the like may be used for topical administration. In addition, the compound of the present invention may be prepared in various dosage forms by being mixed with the pharmaceutically acceptable carriers as described above. For example, for oral administration, the compound may be formulated in the form of tablets, troches, capsules, elixirs, suspensions, syrups, wafers, or the like. For injection, the compound may be formulated in the form of unit dosage ampoules or in multiple-dosage forms. In addition, the compound may be formulated into solutions, suspensions, tablets, capsules, sustained-release preparations, or the like.

Meanwhile, examples of carriers, excipients and diluents suitable for formulation include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil. In addition, the composition of the present invention may further contain a filler, an anticoagulant, a lubricant, a wetting agent, a fragrance, an emulsifier, a preservative, or the like.

The routes of administration of the composition according to the present invention include, but are not limited to, oral, intravenous, intramuscular, intra-arterial, intramedullary, intradural, intracardiac, transdermal, subcutaneous, intraperitoneal, intranasal, gastrointestinal, topical, sublingual and intrarectal routes. Oral or parenteral administration is preferred.

In the present invention, “parenteral” includes subcutaneous, transdermal, intravenous, intramuscular, intra-articular, intra-synovial, intrasternal, intradural, intra-lesional and intra-cranial injection or infusion techniques. The pharmaceutical composition of the present invention may also be formulated as suppositories for intrarectal administration.

In the present invention, the term “pharmaceutically effective amount” refers to a sufficient amount of an agent to provide a desired biological result. Said result may be reduction and/or alleviation of a sign, symptom, or cause of a disease, or any other desired alteration of a biological system. For example, an “effective amount” for therapeutic uses is the amount of the composition disclosed in the present invention, which is required to provide a clinically significant reduction in the disease. An appropriate “effective amount” in any individual case may be determined by one of ordinary skill in the art using routine experimentation. Thus, the expression “effective amount” generally refers to an amount in which an active substance has a therapeutic effect. In the case of the present invention, the active substance serves as both an agent for treating cancer and an agent for preventing, alleviating or treating anticancer drug-resistant cancer.

The dose of the composition of the present invention may vary depending on various factors, including the activity of an active substance used, the patient's age, body weight, general health status, sex, and diet, the time of administration, the route of administration, excretion rate, drug combination, and the severity of a particular disease to be prevented or treated. Although the dose of the active substance may vary depending on the patient's condition and body weight, the severity of the disease, the form of drug, and the route and duration of administration, it may be appropriately selected by those skilled in the art. The active substance may be administered at a dose of 0.0001 to 100 mg/kg/day or 0.001 to 100 mg/kg/day. The active substance may be administered once a day or several times a day. The dose does not limit the scope of the present invention in any way. The compound according to the present invention may be formulated as pills, sugar-coated tablets, capsules, liquids, gels, syrups, slurries, or suspensions.

The active substance of the present invention may be used alone or in combination with surgery, radiotherapy, hormone therapy, chemotherapy, and methods that use biological response modifiers.

### Advantageous Effects

The compound represented by Formula 1 according to the present invention, when used in combination with an anticancer drug for anticancer drug-resistant cancer, may exhibit a significantly improved anticancer effect, and is also capable of inducing the same anticancer effect even when the anticancer drug is used in significantly smaller amounts than previously used, thereby reducing side effects caused by taking the anticancer drug. Furthermore, it is capable of effectively preventing, alleviating or treating not only anticancer drug-resistant cancer, but also cancer that recurs or metastasizes after anticancer drug treatment.

### BRIEF DESCRIPTION OF DRAWINGS

FIGS. 1a to 1c show the results of analyzing changes in gene expression in YUMC-S-P1, YUMC-P1-Main, YUMC-P1-Meta, YUMC-P2-Main and YUMC-P2-Meta cell lines by microarray assay according to one example of the present invention.

FIGS. 2a and 2b depict a schematic view of a process of screening candidate compounds targeting SERCA (ATP2A1) protein according to one example of the present invention and show the results of the screening.

FIGS. 3a to 3e graphically show the results of measuring changes in cell viability of cancer cells by drug administration according to one example of the present invention.

FIGS. 4a to 4e graphically show the results of measuring changes in tumor size by drug administration according to one example of the present invention.

FIGS. 5a to 5e graphically show the results of measuring changes in net tumor weight by drug administration according to one example of the present invention.

FIGS. 6a to 6e graphically show the results of measuring changes in total body weight of mice by drug administration according to one example of the present invention.

### MODE FOR INVENTION

### Examples

### Materials and Methods

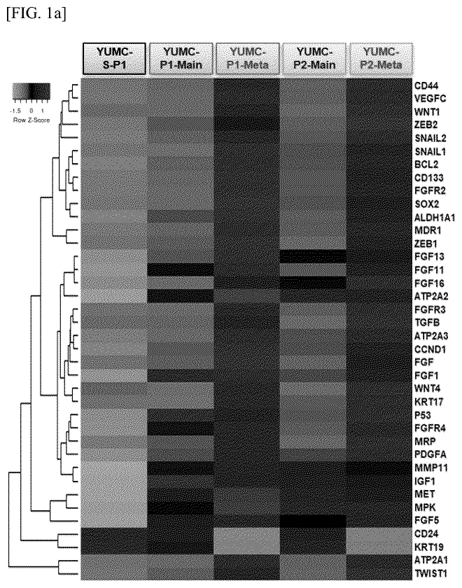

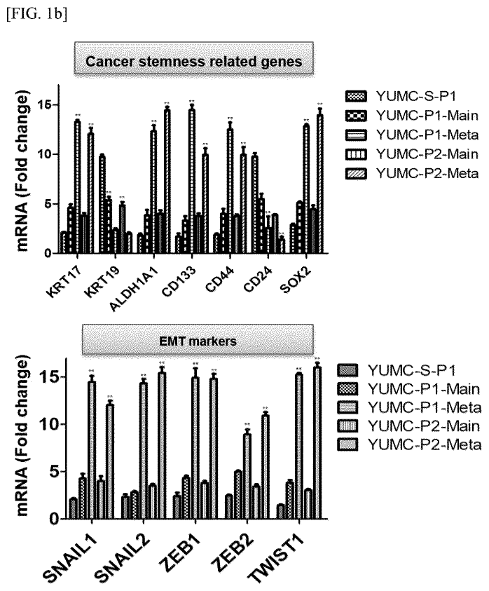

1. mRNA-Seq Data (FIG. 1)

We preprocessed the raw reads from the sequencer to remove low quality and adapter sequences before analysis and aligned the processed reads to the Homo sapiens genome assembly (GRCh37) using HISAT v 2.1.0 (KIM et al, 2015). HISAT utilizes two types of indexes for alignment: a global, whole-genome index, and tens of thousands of small local indexes. Both are constructed using the same Burrows-Wheeler transform (BWT) or graph FM index (GFM) as Bowtie2. Because of the use of these efficient data structures and algorithms, HISAT generates spliced alignments several times faster than Bowtie and the widely used BWA. The reference genome sequence of Homo sapiens (GRCh37) and annotation data were downloaded from the National Center for Biotechnology Information (NCBI). Then, transcript assembly of known transcripts was processed using StringTie v 2.1.3b (Pertea, Mihaela, et al., 2015, 2016). Based on these results, expression abundance of transcript and gene were calculated as read count or fragments per kilobase of exon per million fragments mapped (FPKM) value per sample. The expression profiles were used for additional analyses, such as of differentially expressed genes (DEGs). In groups with different conditions, differentially expressed genes or transcripts were filtered through statistical hypothesis testing.

### 2. Tumor Cell Isolation and Primary Culture (Table 1)

After resection, tumors were kept in phosphate-buffered saline (PBS) with antifungal and antibiotics and moved to the laboratory. Normal tissue and fat were removed and the tissues were rinsed with 1× Hank's Balanced Salt Solution. Tumors were minced in a tube with dissociation medium containing DMEM/F12 with 20% fetal bovine serum supplemented with 1 mg/ml collagenase type IV (Sigma, St. Louis, MO; C5138). Minced and suspended tumor cells were filtered through sterile nylon cell strainers with 70-micron pores (BD Falcon, Franklin Lakes, NJ, USA), rinsed with 50 ml of 1× Hank's Balanced Salt Solution, and centrifuged at 220 g for 5 minutes. Cells were resuspended in RPMI-1640 (Hyclone, South Logan, UT) medium with 10% fetal bovine serum (Hyclone) and 2% penicillin/streptomycin solution (Gibco, Grand Island, NY, USA). Cell viability was determined using the trypan blue dye exclusion method.

### 3. Statistical Analysis of Gene Expression Level

The relative abundances of genes were measured in read count using StringTie. We performed statistical analyses to find differentially expressed genes using the estimates of abundances for each gene in the samples. Genes with one more than zero read count values in the samples were excluded. To facilitate log 2 transformation, 1 was added to each read count value of filtered genes. Filtered data were log 2-transformed and subjected to trimmed mean of M-values (TMM) normalization. The statistical significance of the differential expression data was determined using exactTest, edgeR, and fold change, in which the null hypothesis was that no difference exists among groups. False discovery rate (FDR) was controlled by adjusting the p-value using the Benjamini-Hochberg algorithm. For DEG sets, hierarchical clustering analysis was performed using complete linkage and Euclidean distance as a measure of similarity. Gene-enrichment and functional annotation analysis and pathway analysis for a significant gene list were performed based on Gene Ontology and KEGG pathway analyses.

### 4. Hierarchical Clustering

Hierarchical clustering analysis also was performed using complete linkage and Euclidean distance as a measure of similarity to display the expression patterns of differentially expressed transcripts which are satisfied with |fold change|≥2 and independent t-test raw p<0.05. All data analysis and visualization of differentially expressed genes was conducted using R 3.5.1 (www.r-project.org, accessed on 15 Oct. 2021).

### 5. Cell Viability Assay

Cell viability was measured using the MTT (3-(4,5-Dimethylthiazol-2-yl)-2,5-Diphenyltetrazolium Bromide) assay, cells were seeded in 96-well plates at 7×103 cells per well and incubated overnight to achieve 80% confluency. The detailed protocol can be found in our previous article. Data were expressed as a percentage of the signal observed in vehicle-treated cells and are shown as the means±SEM of triplicate experiments.

### 6. Human PTC Cell Xenograft

All experiments were approved by the Animal Experiment Committee of Yonsei University. YUMC-S-P1, YUMC-P1 and -P2-main or meta patient-derived PTC cells (4.4×106 cells/mouse) were cultured in vitro and then injected subcutaneously into the upper left flank region of female NOD/Shi-scid, IL-2Rγ KOJic (NOG) mice. After 15 days, tumor-bearing mice were grouped randomly (n=10 per group) and treated SERCA inhibitors, thapsigargin (25 mg/kg), candidate 33 (12 mg/kg) and 36 (12 mg/kg, p.o.) with 80 mg/kg sorafenib (p.o.) either alone or combination. Tumor size was measured every three day using calipers. Tumor volume was estimated using the following formula: L×S2/2 (L, longest diameter; S, shortest diameter). Animals were maintained under specific pathogen-free conditions. All experiments were approved by the Animal Experiment Committee of Yonsei University.

### [Results]

### 1. Characteristics of Patient-Derived Drug-Resistant PTC Cell Lines

In this study, anti-cancer drug sensitive or resistant PTC cell lines were generated from tumor specimens collected from patients (Table 1); YUMC-S-P1 (the first cell line developed from patient-derived antineoplastic-sensitive PTC cells), YUMC-P1 or -P2, -main or -meta (first and second cell lines developed from patient-derived main tumor mass or lymph node metastasis PTC cells) cell lines were developed from tumors of patients who received PTC treatment at Severance Hospital, Yonsei University College of Medicine, Seoul, Republic of Korea. YUMC-R-P1-meta and -P2-meta cells resistant to sorafenib and lenvatinib were more aggressive (metastasis) than anti-cancer drug (sorafenib or lenvatinib)-sensitive PTC cells, YUMC-S-P1 (Table 1). To evaluate and compare the genetic differences and signaling pathways activated in both forms of PTC by using YUMC-S-P1, YUMC-P1 and -P2 main or meta cells, we carried out an RNA-sequencing (RNA-Seq)-mediated transcriptome analysis (FIG. 1A).

TABLE 1

| | YUMC-S- | YUMC-P1- | YUMC-P1- | YUMC-P1- | YUMC-P1- |
|---|---|---|---|---|---|
| | P1 | main | meta | main | meta |
| Age at | 53 | 4 | 13 | 4 | 13 |
| Diagnosis | | | | | |
| Gender | Male | Female | Female | Female | Female |
| Primary | Thyroid | Thyroid | Thyroid | Thyroid | Thyroid |
| Disease Site | | | | | |
| Stage | T4aN1bM0 | IVc | IVc | IVc | IVc |
| Primary | Papillary | Papillary | Papillary | Papillary | Papillary |
| Pathology | thyroid | thyroid | thyroid | thyroid | thyroid |
| | cancer | cancer | cancer | cancer | cancer |
| Classification | Fresh | Fresh | Fresh | Fresh | Fresh |
| of specimen | tumor | tumor | tumor | tumor | tumor |
| used for | | | | | |
| culture | | | | | |
| Obtained | Severance | Severance | Severance | Severance | Severance |
| from | Hospital, | Hospital, | Hospital, | Hospital, | Hospital, |
| | Seoul, | Seoul, | Seoul, | Seoul, | Seoul, |
| | Korea | Korea | Korea | Korea | Korea |

RNA-Seq analysis indicated that YUMC-P1-meta and -P2-meta cells were associated with critically higher levels of the expression of epithelial-mesenchymal transition (EMT) markers SNAIL (Zinc finger protein SNAI1), ZEB (Zinc finger E-box-binding homeobox) and TWIST (twist family bHLH transcription factor) when compared with YUMC-S-P1, as illustrated in FIG. 1A-B. Besides TWIST, ZEB, and SNAIL, FGF (Fibroblast growth factor) and FGFR are also highly expressed (FIG. 1B). Cancer stem cell- (CSC, FIG. 1B top), FGF- (FIG. 1B bottom)-associated genes exhibited higher expression levels in YUMC-R-P1-meta and -P2-meta cells than in anti cancer drug-sensitive PTC, YUMC-S-P1. Kyoto Encyclopedia of Genes and Genomes (KEGG) pathway analysis revealed enrichment in cancer stemness-related signaling pathways (Hedgehog, calcium, Wnt, PPAR and TGF/SMAD signaling pathway) in resistant PTC when compared to anti cancer drug-sensitive PTC (FIG. 1C). Furthermore, the calcium signaling pathways were also highly enhanced in sorafenib-resistant PTC cells.

Our results indicate gene pathways related to cancer sternness and SERCA1 expressions are essential for the survival of resistant PTC cells to antineoplastic therapy. Therefore, they can be important targets for developing new anticancer drugs against metastatic forms of metastatic PTC. However, their efficacy must be assessed in clinical trials.

### 2. Discovery of CKP1 and CKP2 as Specific Inhibitors of SERCA1 Using in Silico Screening

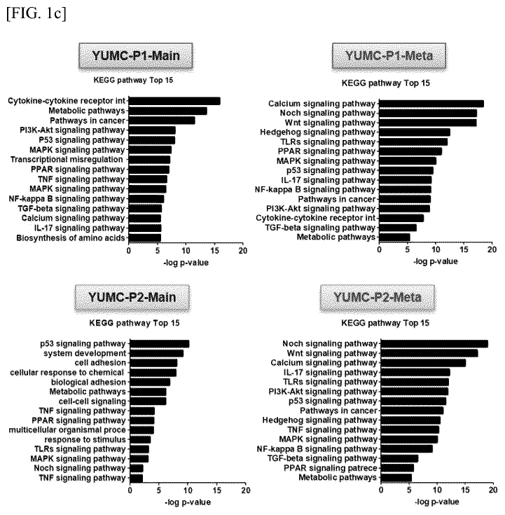

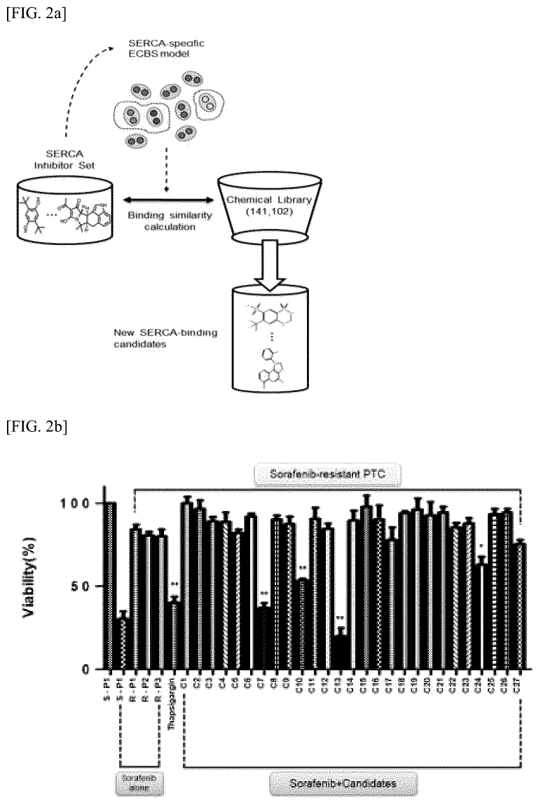

We hypothesized that the functional inhibition of SERCA might be an effective clinical approach for treating sorafenib-resistant PTC. We searched for SERCA1-binding compounds and used in silico screening and pharmacophore modelling to analyze their binding modes to the target. For screening, we used a novel evolutionary chemical binding similarity (ECBS) method based on a classification similarity-learning framework defined with paired chemical data and target's evolutionary relationship. Consequently, 1423 (assessed by using the docking score), 67 (selected manually), and eventually 27 candidate compounds (that included natural compounds) were identified. Of these, 27 candidate compounds had high binding affinity for SERCA, CKP1[Formula 2 below, Molecular Weight: 292.3, Molecular Formula: C19H17ClN2] and CKP2[Formula 3 below, Molecular Weight: 292.3, Molecular Formula: C19H17ClN2](FIGS. 2a and 2b).

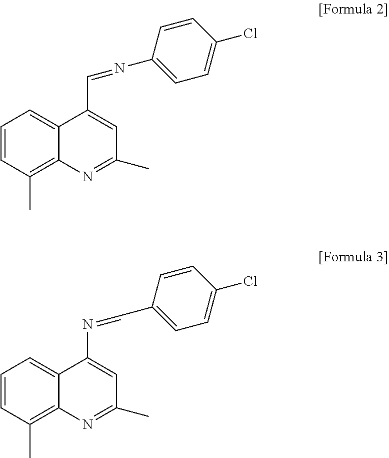 [SMILES: Cc1cc(C=Nc2ccc(Cl)cc2)c2cccc(C)c2n1 ; Cc1cc(N=Cc2ccc(Cl)cc2)c2cccc(C)c2n1]

### 3. Novel Pharmacophore CKP1 and CKP2, Functional SERCA1 Inhibitors, Suppressed the Survival of Metastatic PTC Cells

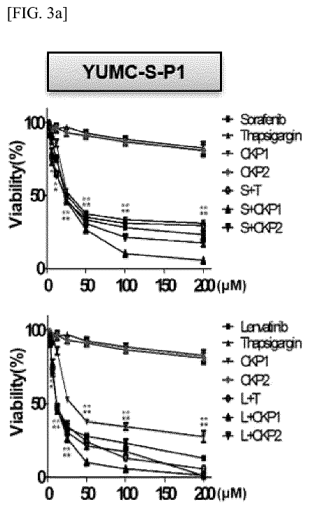

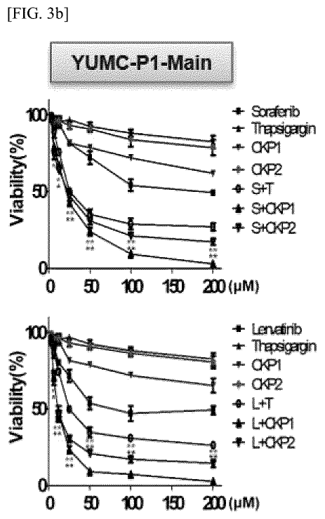

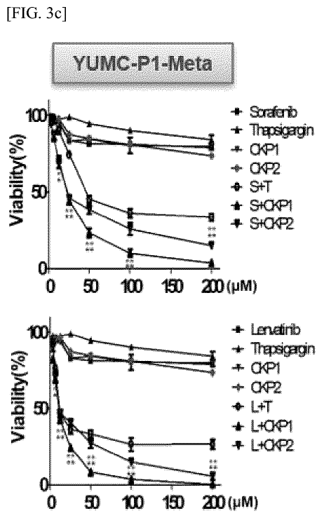

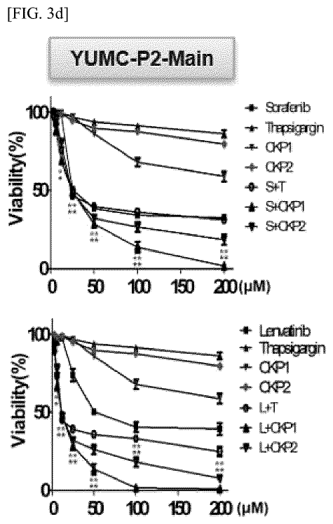

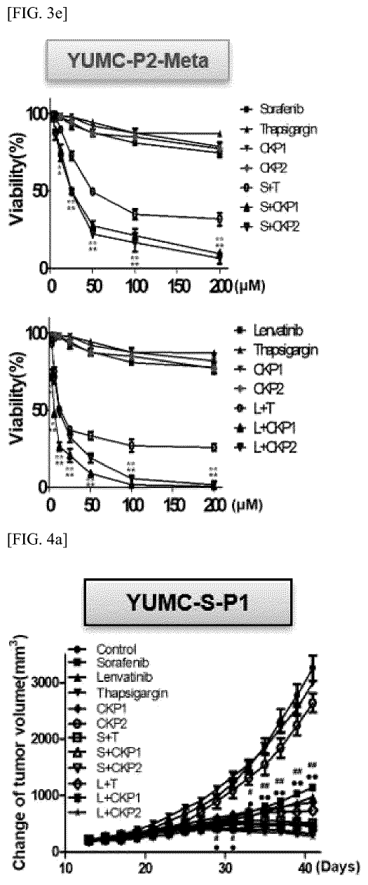

To evaluate to the anti-cancer efficacy of CKP1 and 2, combinatorial strategies with sorafenib or lenvatinib, we carried out cell viability assays for anti-cancer drug-sensitive (YUMC-S-P1) and main mass or lymph node metastasis PTC (YUMC-P1-main or YUMC-P2-main or YUMC-P1-meta or YUMC-P2-meta) cells treated with sorafenib or levatinib each agent treated alone or with CKP1 and CKP2. The viability of YUMC-S-P1, anti-cancer drug-sensitive PTC cells and YUMC-P1-main or YUMC-P2-main cells significantly decreased in a dose-dependent method following sorafenib and lenvatinib administration with or without CKP1 and CKP2 (FIGS. 3a, 3b and 3d). Though the viabilities of metastatic PTC (YUMC-P1-meta or YUMC-P2-meta) cells were no remarkably influenced sorafenib or lenvatinib treatment respectively. In particular, thapsigargin is known as SERCA inhibitor (positive control) or CKP1 and 2, new candidates of SERCA1 inhibitors, combinatorial with sorafenib or lenvatinib treatment remarkably restrained the viability of metastatic PTC cells in a dose-dependent way (FIGS. 3c and 3e). Furthermore, thapsigargin, known as SERCA inhibitor or CKP 1 or 2 alone treatment did not critical influence to viability of whole PTC cells.

These results indicated that SERCA could be essential factor in prolonged metastatic PTC cell survival when treatment with several chemotherapies by handling overloaded cytosolic free calcium restoration.

4. In Vivo Assessment of the Anticancer Efficacy of CKP 1 and 2 in a Patient-Derived metastatic PTC Cell Mouse Xenograft Model

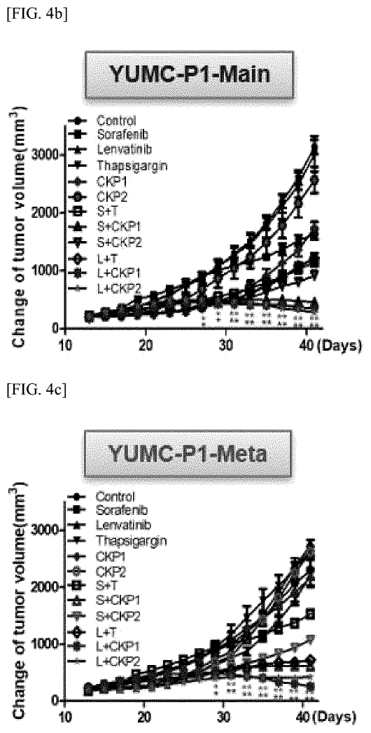

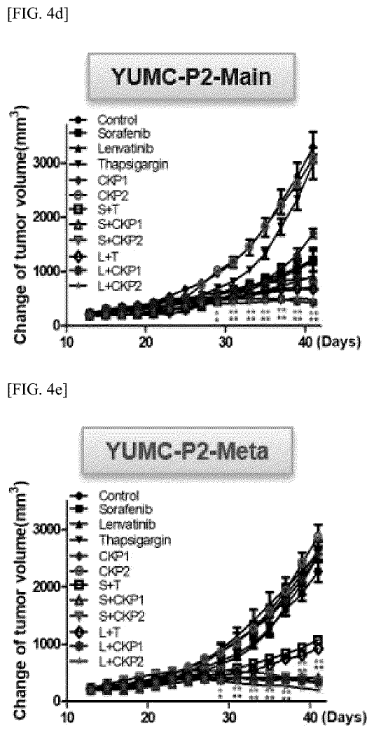

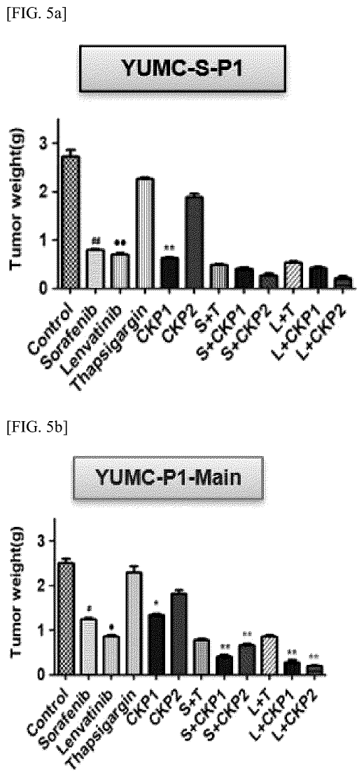

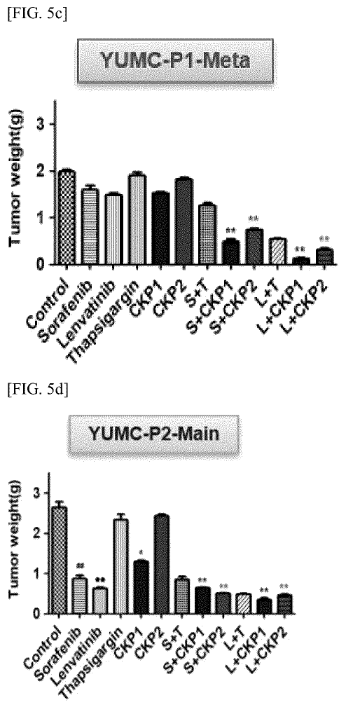

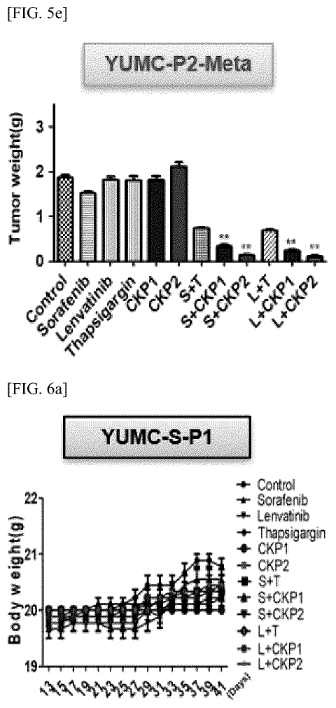

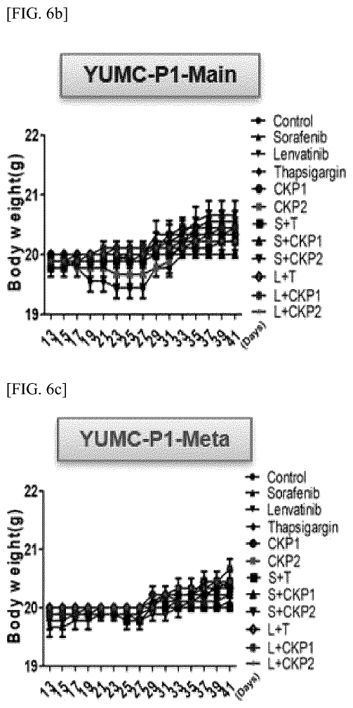

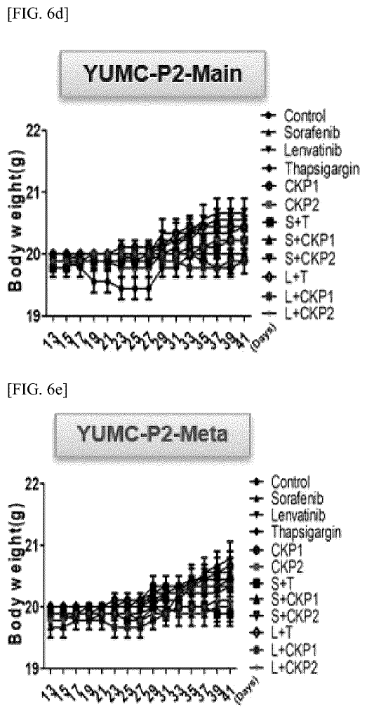

We developed mouse xenograft models using anti cancer drug-sensitive (YUMC-S-P1) and metastatic PTC (YUMC-P1 and -P2-meta) cells and evaluated the anticancer effect of monotherapy with sorafenib, lenvatinib or with SERCA inhibitors (thapsigargin, CKP1 and 2) as well as that of the combination therapy between sorafenib or lenvatinib and a SERCA inhibitor. In the xenograft model developed using anti cancer drug sensitive PTC cells, sorafenib or lenvatinib administration with or without SERCA inhibitors decreased significantly tumor size (FIG. 4a to 4e). No significant changes in resected tumor weight were found between any of the treatment groups (FIG. 5a to 5e). None of the therapy regimens did significantly influence mouse body weight (FIG. 6a to 6e). Nonetheless, in xenograft model developed using metastatic PTC cells (YUMC-P1 or -P2-meta), monotherapy with either sorafenib and lenvatinib did not induce significant tumor shrinkage. However, combination with SERCA inhibitors resulted in substantial tumor shrinkage. Similar results were observed for the weight of the resected tumor. None of the treatment regimens did significantly influence mouse body weight.

Taken together, these results showed that novel SERCA inhibitors, CKP1 and 2 could provide a novel clinical approach for treating patients with metastatic PTC as they induce significant tumor shrinkage in a xenograft tumor model developed using patient-derived antineoplastic-resistant cells.

1. A method for enhancing sensitivity of a subject to an anticancer drug, comprising administering to the subject a pharmaceutical composition containing as an active ingredient a compound represented by the following Formula 1 or a pharmaceutically acceptable salt thereof as an active ingredient: 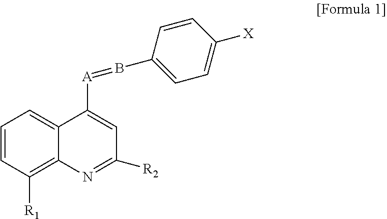
wherein
A and B are each independently —CH or N;
R₁ and R₂ are each independently selected from the group consisting of hydrogen and C₁-C₅ alkyl; and
X is hydrogen or halogen.

2. The method according to claim 1, wherein
A is —CH;
B is N;
R₁ and R₂ are each independently C₁ to C₃ alkyl; and
X is halogen, or
wherein
A is N;
B is —CH;
R₁ and R₂ are each independently selected from the group consisting of C₁ to C₃ alkyl; and
X is halogen.

3. The method according to claim 1, wherein the cancer is resistant, recurrent or metastatic cancer, preferably wherein the cancer is one or more cancers selected from the group consisting of thyroid cancer, colorectal cancer, breast cancer, uterine cancer, fallopian tube cancer, ovarian cancer, gastric cancer, brain cancer, rectal cancer, small intestine cancer, esophagus cancer, lymph node cancer, gallbladder cancer, lung cancer, skin cancer, kidney cancer, bladder cancer, blood cancer, pancreatic cancer, prostate cancer, endocrine gland cancer, oral cancer, and liver cancer.

4. The method according to claim 1, wherein the compound is administered in combination with the anticancer drug to enhance an activity of the anticancer drug.

5. The method according to claim 4, wherein the anticancer drug is at least one selected from the group consisting of lenvatinib, sorafenib, imatinib, erlotinib, neratinib, lapatinib, gefitinib, vandetanib, nilotinib, semasanib, bosutinib, axitinib, cediranib, regorafenib, lestaurtinib, asciminib, ibrutinib, sunitinib, afatinib, dasatinib, nitrogen mustard, oxaliplatin, rituximab, panitumumab, trastuzumab, bortezomib, carboplatin, bevacizumab, cisplatin, cetuximab, aflibercept, Viscum album, asparaginase, hydroxycarbamide, estramustine, gemtuzumab ozogamicin, ibritumomab tiuxetan, heptaplatin, methylaminolevulinic acid, amsacrine, alemtuzumab, procarbazine, alprostadil, holmium nitrate chitosan, gemcitabine, doxifluridine, pemetrexed, tegafur, capecitabine, gimeracin, oteracil, azacitidine, methotrexate, cytarabine, fluorouracil, fludarabine, enocitabine, flutamide, decitabine, mercaptopurine, thioguanine, cladribine, leucovorin, carmophor, raltitrexed, interferon alpha-2a, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, pepromycin, temsirolimus, temozolomide, busulfan, ifosfamide, cyclophosphamide, melphalan, altretamine, dacarbazine, thiotepa, nimustine, chlorambucil, mitolactol, leucovorin, tretonin, exemestane, amino glutesimide, anagrelide, navelbine, fadrazole, tamoxifen, toremifene, testolactone, anastrozole, letrozole, vorozol, bicalutamide, lomustine, and carmustine.

6. The method according to claim 1, wherein the pharmaceutical composition is administered in combination with a food.

7. The method according to claim 6, wherein the pharmaceutical composition is also administered in combination with an anticancer drug to enhance an activity of the anticancer drug.

8. The method according to claim 7, wherein the anticancer drug is at least one selected from the group consisting of lenvatinib, sorafenib, imatinib, erlotinib, neratinib, lapatinib, gefitinib, vandetanib, nilotinib, semasanib, bosutinib, axitinib, cediranib, regorafenib, lestaurtinib, asciminib, ibrutinib, sunitinib, afatinib, dasatinib, nitrogen mustard, oxaliplatin, rituximab, panitumumab, trastuzumab, bortezomib, carboplatin, bevacizumab, cisplatin, cetuximab, aflibercept, Viscum album, asparaginase, hydroxycarbamide, estramustine, gemtuzumab ozogamicin, ibritumomab tiuxetan, heptaplatin, methylaminolevulinic acid, amsacrine, alemtuzumab, procarbazine, alprostadil, holmium nitrate chitosan, gemcitabine, doxifluridine, pemetrexed, tegafur, capecitabine, gimeracin, oteracil, azacitidine, methotrexate, cytarabine, fluorouracil, fludarabine, enocitabine, flutamide, decitabine, mercaptopurine, thioguanine, cladribine, leucovorin, carmophor, raltitrexed, interferon alpha-2a, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, pepromycin, temsirolimus, temozolomide, busulfan, ifosfamide, cyclophosphamide, melphalan, altretamine, dacarbazine, thiotepa, nimustine, chlorambucil, mitolactol, leucovorin, tretonin, exemestane, amino glutesimide, anagrelide, navelbine, fadrazole, tamoxifen, toremifene, testolactone, anastrozole, letrozole, vorozol, bicalutamide, lomustine, and carmustine.

9. A pharmaceutical composition for preventing or treating cancer, the pharmaceutical composition containing a compound represented by the following Formula 1 or a pharmaceutically acceptable salt thereof as an active ingredient: 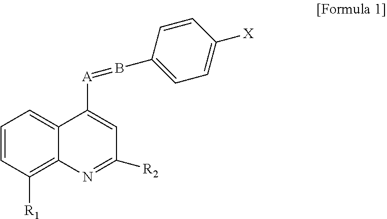
wherein
A and B are each independently —CH or N;
R₁ and R₂ are each independently selected from the group consisting of hydrogen and C₁-C₅ alkyl; and
X is hydrogen or halogen.

10. The pharmaceutical composition according to claim 9, wherein
A is —CH;
B is N;
R₁ and R₂ are each independently selected from the group consisting of C₁ to C₃ alkyl; and
X is halogen, or wherein
A is N;
B is —CH;
R₁ and R₂ are each independently selected from the group consisting of C₁ to C₃ alkyl; and
X is halogen.

11. The pharmaceutical composition according to claim 9, further containing at least one anticancer drug selected from the group consisting of lenvatinib, sorafenib, imatinib, erlotinib, neratinib, lapatinib, gefitinib, vandetanib, nilotinib, semasanib, bosutinib, axitinib, cediranib, regorafenib, lestaurtinib, asciminib, ibrutinib, sunitinib, afatinib, dasatinib, nitrogen mustard, oxaliplatin, rituximab, panitumumab, trastuzumab, bortezomib, carboplatin, bevacizumab, cisplatin, cetuximab, aflibercept, Viscum album, asparaginase, hydroxycarbamide, estramustine, gemtuzumab ozogamicin, ibritumomab tiuxetan, heptaplatin, methylaminolevulinic acid, amsacrine, alemtuzumab, procarbazine, alprostadil, holmium nitrate chitosan, gemcitabine, doxifluridine, pemetrexed, tegafur, capecitabine, gimeracin, oteracil, azacitidine, methotrexate, cytarabine, fluorouracil, fludarabine, enocitabine, flutamide, decitabine, mercaptopurine, thioguanine, cladribine, leucovorin, carmophor, raltitrexed, interferon alpha-2a, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, pepromycin, temsirolimus, temozolomide, busulfan, ifosfamide, cyclophosphamide, melphalan, altretamine, dacarbazine, thiotepa, nimustine, chlorambucil, mitolactol, leucovorin, tretonin, exemestane, amino glutesimide, anagrelide, navelbine, fadrazole, tamoxifen, toremifene, testolactone, anastrozole, letrozole, vorozol, bicalutamide, lomustine, and carmustine.

12. The pharmaceutical composition according to claim 11, wherein the cancer is resistant, recurrent or metastatic cancer, preferably wherein the cancer is resistant to at least one anticancer drug selected from the group consisting of lenvatinib, sorafenib, imatinib, erlotinib, neratinib, lapatinib, gefitinib, vandetanib, nilotinib, semasanib, bosutinib, axitinib, cediranib, regorafenib, lestaurtinib, asciminib, ibrutinib, sunitinib, afatinib, and dasatinib.

13. The pharmaceutical composition according to claim 11, wherein the cancer is any one or more selected from the group consisting of thyroid cancer, colorectal cancer, breast cancer, uterine cancer, fallopian tube cancer, ovarian cancer, gastric cancer, brain cancer, rectal cancer, small intestine cancer, esophagus cancer, lymph node cancer, gallbladder cancer, lung cancer, skin cancer, kidney cancer, bladder cancer, blood cancer, pancreatic cancer, prostate cancer, endocrine gland cancer, oral cancer, and liver cancer.

14. A pharmaceutical composition for enhancing sensitivity to anticancer drug treatment, the pharmaceutical composition containing a compound represented by the following Formula 1 or a pharmaceutically acceptable salt thereof as an active ingredient: 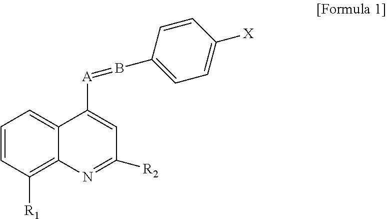
wherein
A and B are each independently —CH or N;
R₁ and R₂ are each independently selected from the group consisting of hydrogen and C₁-C₅ alkyl; and
X is hydrogen or halogen.

15. The pharmaceutical composition according to claim 14, wherein the anticancer drug is at least one selected from the group consisting of lenvatinib, sorafenib, imatinib, erlotinib, neratinib, lapatinib, gefitinib, vandetanib, nilotinib, semasanib, bosutinib, axitinib, cediranib, regorafenib, lestaurtinib, asciminib, ibrutinib, sunitinib, afatinib, dasatinib, nitrogen mustard, oxaliplatin, rituximab, panitumumab, trastuzumab, bortezomib, carboplatin, bevacizumab, cisplatin, cetuximab, aflibercept, Viscum album, asparaginase, hydroxycarbamide, estramustine, gemtuzumab ozogamicin, ibritumomab tiuxetan, heptaplatin, methylaminolevulinic acid, amsacrine, alemtuzumab, procarbazine, alprostadil, holmium nitrate chitosan, gemcitabine, doxifluridine, pemetrexed, tegafur, capecitabine, gimeracin, oteracil, azacitidine, methotrexate, cytarabine, fluorouracil, fludarabine, enocitabine, flutamide, decitabine, mercaptopurine, thioguanine, cladribine, leucovorin, carmophor, raltitrexed, interferon alpha-2a, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleomycin, daunorubicin, dactinomycin, pirarubicin, aclarubicin, pepromycin, temsirolimus, temozolomide, busulfan, ifosfamide, cyclophosphamide, melphalan, altretamine, dacarbazine, thiotepa, nimustine, chlorambucil, mitolactol, leucovorin, tretonin, exemestane, amino glutesimide, anagrelide, navelbine, fadrazole, tamoxifen, toremifene, testolactone, anastrozole, letrozole, vorozol, bicalutamide, lomustine, and carmustine.
